(19) United States
(12) Patent Application Publication
(10) Pub. No.: US 2001/0041803 A1
(43) Pub. Date: Nov. 15, 2001

(54) Conversion of 9-dihydro-13-acetylbaccatin III to baccatin III and 10-deacetyl baccatin III

(76) Inventors: Gertrude C. Kasitu, Debert (CA); Japheth W. Noah, Debert (CA)

Correspondence Address: Scott D. Rothenberger, Dorsey & Whitney LLP, 220 South Sixth Street, Minneapolis, MN, 55402-1498, US

(21) Appl. No.: 09/811,690

(22) Filed: Mar. 19, 2001

Related U.S. Application Data
(60) Provisional application No. 60/190,995, filed on Mar. 21, 2000.

(51) Int. Cl. C 07D 3 5/14; C08F 8/12

(52) U.S. Cl. 549/510; 525/383

(57) ABSTRACT

Novel methods and synthetic intermediates to prepare baccatin III and 10-deacetylbaccatin from readily available 9-dihydro-13-acetylbaccatin III are described. Selective protection and deprotection of the C-7 hydroxyl functionality provides an entry into facile synthesis of novel taxol intermediates, as well as, providing new methods for the preparation of paclitaxel and docetaxel in large scale. Selective oxidation of the C-9 hydroxyl without the need for protection of the C-7 hydroxyl is described.

### CROSS-REFERENCE TO RELATED APPLICATIONS

This application claims priority to U.S. Provisional Application Ser. No. 60/190,995, filed Mar. 21, 2000, entitled “Conversion of 9-Dihydro-13-acetylbaccatin III to Baccatin III and 10-Deacetylbaccatin III” by Gertrude C. Kasitu and Japheth W. Noah, the contents of which are incorporated herein by reference in their entirety.

### BACKGROUND OF THE INVENTION

Few molecules have attracted so much multidisciplinary research efforts as has Paclitaxel (TAXOL®), the compound having the formula 1, since its discovery four decades ago.

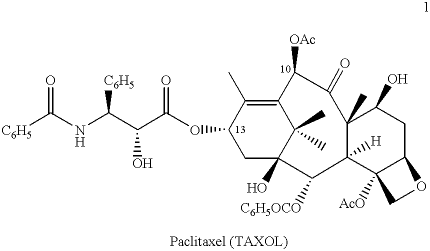

TAXOL® 1 and its synthetic analogue, TAXOTERE®, the compound having the formula 2, are clinically useful in the treatment of ovarian and breast cancer. TAXOL® has been approved most recently for treatment of AIDS-related Kaposi's Sarcoma.

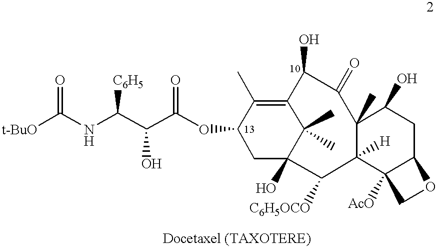

Paclitaxel was first isolated from the bark of the pacific yew, Taxus brevigolia (Wani et al., J. Am. Chem. Soc., 1971, 93, 2325-2327). Naturally occurring paclitaxel is in limited quantities and cannot meet the potential demand for therapeutic application. The limited supply of paclitaxel has restricted promising new drug developments.

As a consequence of the limited supply of naturally occurring paclitaxel, strategies to increase the supply of paclitaxel by other means have been adopted. These include cell culture, total synthesis from simple starting materials, and semi-synthesis from readily available natural taxane derivatives. Although production via cell culture is very promising, the process to date has not reached large scale commercialization. The total synthesis of paclitaxel has been accomplished by a number of researchers (Holton; J. Am. Chem. Soc., 1994, 116, 1597 & 1599, J. Am. Chem. Soc., 1988, 110, 6558, Nicolaou; J. Am. Chem. Soc. 1995, 117, 653 and references cited therein, Danishefsky; J. Am. Chem. Soc., 1996, 118, 2843, Mukaiyama; Chem. Eur. J., 1999, 5, 121-161) however, none of the synthetic processes are practical commercially. The drawbacks of total synthesis include poor overall yields and lengthy complicated synthetic steps.

The central structural unit of paclitaxel is baccatin III, a diterpenoid having the chemical structure 4:

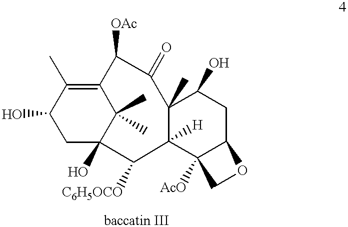

Baccatin III is also very similar in structure to 10-deacetylbaccatin III (“10-DAB”), which has the chemical structure 3:

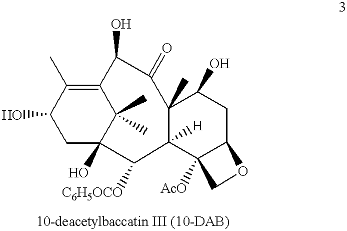

but which lacks an acetate ester at the 10-position alcohol.

10-DAB, 3, is a starting material for the semi-synthesis of paclitaxel and taxotere, and can be readily extracted from the needles and twigs of the European Yew tree, Taxus baccata.

However, baccatin III, 10-DAB and other taxane compounds, do not, exhibit the degree of anti-tumor activity demonstrated by paclitaxel. Accordingly, the semi-synthesis of paclitaxel from baccatin III, 10-DAB and other taxane compounds is of great interest and importance.

The basic taxane structure of baccatin III and 10-DAB have the carbon skeletons of paclitaxel/docetaxel without the side chain at the C-13 position. The basic diterpene structure of baccatin III and 10-DAB are viewed as important starting materials in paclitaxel/docetaxel semisythesis and their importance is expected to increase as therapeutic applications increase. It already appears that baccatin III and 10-DAB will be useful starting materials for the preparation of second and third generation taxol-like compounds.

Therefore, a need exists for a facile semi-synthesis of low cost and high efficiency for the preparation of paclitaxel derivatives and intermediates such as baccatin III and 10-DAB.

### SUMMARY OF THE INVENTION

The present invention is drawn to novel methods for the preparation of 10-deacetylbaccatin III (10-DAB), 3, and baccatin III, 4, and their analogues, as useful intermediates for the preparation of docetaxel, 2, and paclitaxel, 1, respectively and analogues thereof. The present invention provides the advantage that starting material for the preparation of intermediates, 9-dihydro-13-acetylbaccatin III, compound 5 is abundant in the needles of the Eastern yew, Taxus canadensis. Isolating 9-dihydro-13-acetylbaccatin III from the needles, a renewable source, is more friendly environmentally than isolating from the bark.

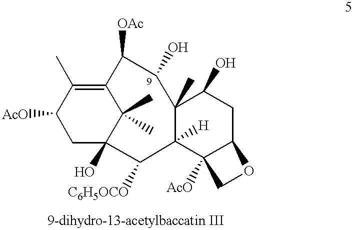

The synthetic preparations provided by the invention are economical and provide overall yields of between about 65 and 70% of the intermediates 3 and 4. The simple and elegant method of conversion from 9-dihydro-13-acetylbaccatin III, 5, to 10-DAB, 3, or baccatin III, 4, provided by the invention affords low cost highly efficient methods to produce these useful drug intermediates and analogues thereof. Thus the methods of the invention provide an entry into the efficient preparation of paclitaxel, 1, and docetaxel, 2, and analogues thereof, previously hindered by the lack of readily available starting materials.

In one embodiment, the present invention provides a method for the preparation of useful intermediates for the semi-synthesis of 10-deacetylbaccatin III (10-DAB), 3, and baccatin III, 4, and analogues thereof from 9-dihydro-13-acetylbaccatin III, compound 5. The method includes the steps of selectively protecting the C-7 hydroxyl group of 9-dihydro-13-acetylbaccatin III to provide compound 6:

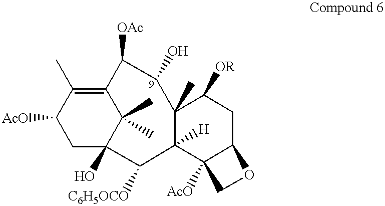

wherein R is a protecting group, as defined below. Preferably, R is acetyl, tesyl or methoxybenzyl. Selective oxidation of the C-9 hydroxyl group affords intermediate 7:

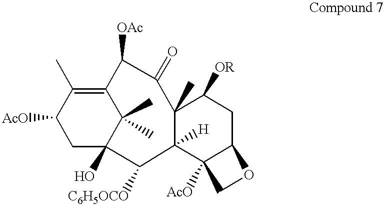

which is a useful intermediate on the synthetic path to baccatin III, compound 4 and 10-DAB, compound 3. In one embodiment, selective deprotection of the C-7 and C-13 protected hydroxyl groups in compound 7 provides baccatin III, compound 4. Alternatively, selective deprotection of the C-7, C-10 and C-13 hydroxyl groups in compound 7 after oxidation provides 10-DAB, compound 3. In general, each step of the method, e.g., protection, oxidation, deprotection, occurs in greater than 80% isolated yield, preferably in greater than 90% isolated yield, and most preferably greater than 95% isolated yield.

Surprisingly, it was discovered that the C-9 hydroxyl group of 9-dihydro-13-acetylbaccatin III, compound 5, can be selectively oxidized by treatment with carefully identified oxidizing reagents such as TPAP/NMO, IBX, polymeric TEMPO or polyethyleneglycol-methylsulfoxide at room temperature to afford intermediate compound 8 without prior protection of the C-7 hydroxyl group.

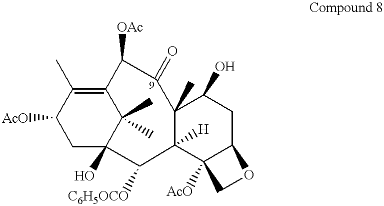

Subsequent conversion of the C-13 acetate group into a hydroxyl group can be effected by treatment of compound 8 with methyllithium in tetrahydrofuran or lithium hydroxide in aqueous methanol or methanolic potassium carbonate to provide baccatin III, compound 4. Alternatively, intermediate compound 8 can be treated with hydrazine monohydrate in ethanol to hydrolyze the acetate protected hydroxyl groups at C-10 and C-13 to provide 10-DAB, compound 3.

### DETAILED DESCRIPTION OF THE INVENTION

The features and other details of the invention will now be more particularly described and pointed out in the claims. It will be understood that the particular embodiments of the invention are shown by way of illustration and not as limitations of the invention. The principle features of this invention can be employed in various embodiments without departing from the scope of the invention.

The present invention is drawn to novel methods for the preparation of 10-deacetylbaccatin III (10-DAB), 3, and baccatin III, 4, and analogues thereof, as useful intermediates for the preparation of docetaxel, 2, and paclitaxel, 1, and their analogues, respectively from the taxane, 9-dihydo-13-acetyl-baccatin III, 5. The present invention provides the advantage that starting material for the preparation of the intermediates is readily available from an abundant source, 9-dihydro-13-acetylbaccatin III, compound 5, isolated from the needles of the Eastern yew, Taxus canadensis. Synthetic manipulation of compound 5, affords useful intermediates for the preparation of 10-deacetylbaccatin III (10-DAB), 3, and baccatin III, 4, and analogues thereof as described herein.

The term “taxane” refers to compounds having the tricyclic ring represented by the following formula:

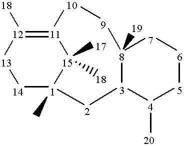

The chemical structure of taxanes and related compounds is described in Gueritte-Voegelin J. Nat. Prod. 50:9-18 (1987).

9-dihydro-13-acetylbaccatin III, compound 5, can be isolated by alcoholic extraction from the crushed needles and twigs of Taxus canadensis. The extract can be purified by separation techniques known by those of ordinary skill in the art, starting with partitioning with solvent systems of acetone, methanol, hexane, heptane and water to remove fats and lipids. The defatted crude extract is then partitioned between solvent systems of methanol, methylene chloride, chloroform, ethyl acetate and water. The methylene chloride or chloroform and ethyl acetate extraction layers contain compound 5. Further purification can be accomplished by planet coil countercurrent chromatography (PCCC), using solvent systems of hexane, methanol, methylene chloride, chloroform, toluene and water or suitable aqueous buffer solutions. Representative extraction procedures are outlined in PCT/US93/03532, filed Apr. 14, 1993 by P. Gunawardana et al., U.S. Pat. Nos. 5,352,806, 5,900,367, 5,969,165, 5,969,752, 6,002,025 and Canadian applications 2,203,844 and 2,213,952, the contents of which are expressly incorporated herein by reference.

In one embodiment, the present invention provides a method for the preparation of useful intermediates for the preparation of 10-deacetylbaccatin III (10-DAB), 3, and baccatin III, 4, and analogues thereof from 9-dihydro-13-acetylbaccatin III, compound 5. The method includes the steps of selectively protecting the C-7 hydroxyl group of 9-dihydro-13-acetylbaccatin III, selective oxidation of the C-9 hydroxyl group, and selective deprotection of the C-7, C-10 and C-13 hydroxyl groups to provide baccatin III, compound 4. Preferably, the methods of the invention include the use of polymers as protecting groups in solid phase or liquid synthesis. Use of polymers as protecting groups provides that the synthetic steps do not require chromatography, but only filtration and concentration of reactants. Furthermore, advantageously, the polymeric protecting group(s) can be regenerated and recycled (green chemistry).

Selective deprotection of the C-7 hydroxyl and C-10 hydroxyl groups after oxidation provides 10-DAB, compound 3. In general, each step of the method, e.g., protection, oxidation, deprotection, occurs in greater than 80% isolated yield, preferably in greater than 90% isolated yield, and most preferably greater than 95% isolated yield.

For example, 10-deacetylbaccatin III (10-DAB), 3, and baccatin III, 4 can be prepared by the following method depicted in Scheme 1:

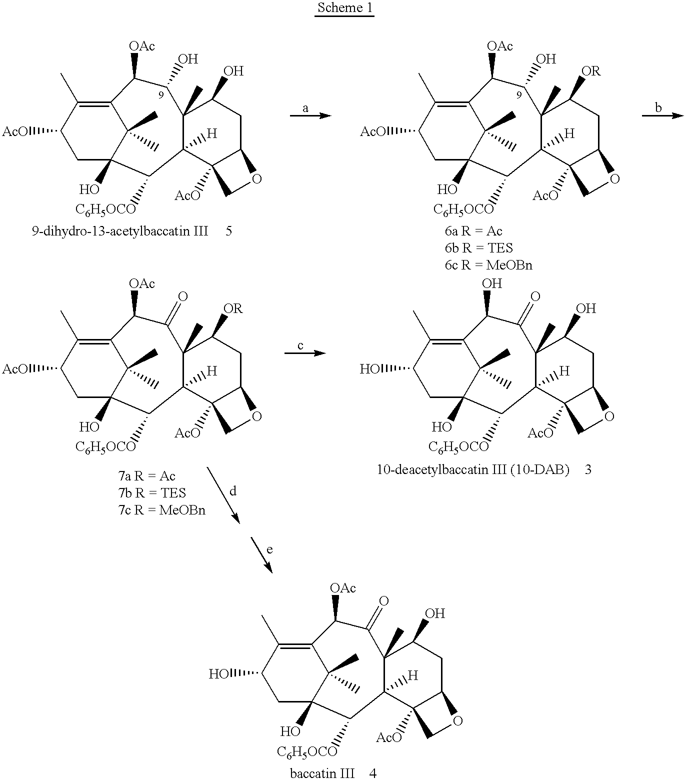

wherein R is generally defined as a protecting group, preferably acetate or a polymeric protecting group as generally defined herein.

The term “protecting group” is a term well known in the art and relates to functional groups of compounds which can undergo chemical transformations which prevent undesired reactions and/or degradations during synthesis. Suitable protecting groups are found in T. W. Greene, “Protective Groups in Organic Synthesis,” John Wiley & Sons 3^{rd} Ed. (1999), the contents of which are incorporated herein by reference. For example, suitable protecting groups include acyl groups, e.g., acetate (Ac), silyl protecting groups, e.g., tesyl (TES), aromatic ethers, e.g., P-methoxybenzyl (PMP). Moreoever, suitable and preferred protecting groups include polymeric protecting groups such as O—Si-diethylbutyl-polymer bound, or O-acetyl-polymer bound or O-tritylpolymer bound.

The present invenon provides the advantage that use of acetate, in particular, as well as other protecting groups that are much more efficient, e.g., higher yields, less time, less by-products, in protecting the C-7 hydroxyl group of 9-dihydro-13-actylbaccatin III than known tesyl protection chemistry (See Canadian Application 2,188,190 by Lolita Zamir et al., Oct. 18, 1996). For example, the yields for acetylation of the C-7 hydroxyl, oxidation of the C-9 hydroxyl, and deacetylation of the C-7 acetate proceed in greater than 90%, 100% and greater than 85% yields, respectively, affording an overall yield of greater than 75%. The process is adapatable for industrial scale production. The acetylation takes less than 15 minutes for completion, the oxidation less than 30 minutes at quantitative yields, e.g., TPAP, polymeric TPAP, IBX, TEMPO, polymeric TEMPO, etc. as disclosed herein, and deacetylation, less than 3 hours (For suitable reaction conditions with IBX, see, for example, K. C. Nicolou et al, J.Am.Chem.Soc. 2000,7596; E. J. Corey et al, Tetrahedron Lett. (1995), 3488; M. Frigerio et al, Tetrahedron Lett. (1994), 8019, ibid. J.Org. Chem.1999,4538.). Pure 10 DAB-III is obtained in under a day under mild conditions, e.g, at room temperature. The yields and ease of synthesis is surprising in view of the tesylation chemistry as described below. Additionally, acetic anhydride is an inexpensive, easy to handle, readily available material in contrast to the more expensive tesylchloride which is difficult to handle in large scale quantities.

Protection of the C-7 hydroxyl in 9-dihydro-13-acetylbaccatin III with tesylation chemistry results in yields of about 60% of 9-dihydro-13-acetyl-7-tesylbaccatin III. The reaction generally requires at least 24 hours to convert the C-7 hyroxyl to this 60% conversion level. A disadvantage of this chemistry is that a by product, 13-tesyl-9-dihydro-7-tesylbaccatin III is generated. Additionally, an intermediate chromatographic or separation step is required to isolate the mono-tesylated product, 9-dihydro-13-acetyl-7-tesylbaccatin III.

Referring to Scheme 1, in one exemplary method, step a) includes protection of the C-7 hydroxyl group of 9-dihydro-13-acetylbaccatin III, compound 5, by treatment with acetic anhydride (Ac₂O) and DMAP (p-dimethylamino pyridine) in methylene chloride to yield the C-7 acetate 6a. In step b), the C-9 unprotected hydroxyl group in the C-7 acetate 6a is oxidized by reaction with TPAP (tetrapropylammonium perruthenate), NMO (4-methylmorpholine-N-oxide) and 4A molecular sieves to afford the C-9 oxidized acetate 7a (See for example, S. V. Ley et al. (1998) Journal of Chemical Soc. Perkin Trans. 1, 2239 and B. Hinzen & S. V. Ley (1998), J. Chem. Soc. Perkin Trans 1, 1). The C-9 oxidized acetate can then be treated with hydrazine (NH₂NH₂) in ethanol or methanolic potassium carbonate to provide 10-DAB, compound 3. Alternatively, the intermediate 7a can be converted to baccatin III, compound 4, by controlled treatment with potassium carbonate in methanol.

In a second exemplary method, step a) includes protection of the C-7 hydroxyl group of 9-dihydro-13-acetylbaccatin III, compound 5, by treatment with TESCl (triethylsilyl chloride) and an amine base such as triethylamine in THF, pyridine or imidazole in DMF to yield the C-7 tesyl protected hydroxyl 6b. In step b), the C-9 unprotected hydroxyl group in the C-7 tesyl protected hydroxyl 6b is oxidized by reaction with TPAP (tetrapropylammonium perruthenate), NMO (4-methylmorpholine-oxide) and 4A molecular sieves to afford the C-9 oxidized acetate 7b. The C-9 oxidized tesyl protected 7b can then be treated with hydrazine in ethanol or methanolic potassium carbonate followed by hydrofluoric acid-pyridine to provide 10-DAB, compound 3. Alternatively, the intermediate 7b can be converted in baccatin III, compound 4, by treatment with methyl lithium followed by hydrofluoric acid-pyridine.

In a third exemplary method, step a) includes protection of the C-7 hydroxyl group of 9-dihydro-13-acetylbaccatin III, compound 5, by treatment with methoxybenzyl alcohol and catalytic ytterbium (III) triflate (Yb(OTf)₃) in dichloromethane to yield the C-7 benzyl protected hydroxyl 6c. In step b), the C-9 unprotected hydroxyl group in the C-7 benzyl protected hydroxyl 6c is oxidized by reaction with TPAP (tetrapropylammonium perruthenate), NMO (4-methylmorpholine-N-oxide) with 4A molecular sieves to afford the C-9 oxidized acetate 7c. The C-9 oxidized benzyl protected 7c can then be treated with hydrazine in ethanol followed by dichlorodicyanoquinone (DDQ) in a mixture of dichloromethane and water to provide 10-DAB, compound 3. Alternatively, the intermediate 7c can be converted to baccatin III, compound 4, by debenzylation with DDQ in dichloromethane-water followed by treatment with methyllithium in THF or lithium hydroxide in aqueous methanol.

In a fourth exemplary method step a) includes protection of C-7 hydroxyl group of 9-dihydro-acetylbaccatin III, compound 5, by treatment with chlorosilyldiethylbutyl polymer bound and imidazole, in DMF for 12 hours. The product was oxidized with TPAP/NMO or TPAP/Oxygen in dichloromethane. The polymeric protecting group was removed by HF-pyridine in dichloromethane. This example is not meant to be limiting. Suitable polymeric silyl protecting agents include those known in the art, such as chlorodimethylsilyl polystyrene (See for example, Y. Tanabe, et al. (1994), Tetrahedron Lett., 35, 8413, Y. Hu et al., (1998), J. Org. Chem., 63, 4518, B. R. Stranix and H. Q. Liu, J.Org.Chem. (1997), 62, 6183, or I. Hirao et al, Tetrahedron Letters (1998) 2989.) and SEMCl (see for example, B. H. Lipshutz et al, Tetrahedron Lett. (1980), 3343).

In a fifth exemplary method step a) includes protection of C-7 hydroxyl group of 9-dihydro-acetylbaccatin III, compound 5, by treatment with acetyl bound polymer and a weak base, in DMF for 12 hours. The product was oxidized with TPAP/NMO or TPAP/Oxygen in dichloromethane. The polymeric protecting group was removed by dilute acid. Suitable exemplary references include A. Routledge et al., Syn Lett, 61, S. Kobayashi et al., Tetrahedron Letter (1999), 1341, C. C. Lenzoff et al. , Can J. Chem. (2000), and references cited therein, and H. J. Meyers et al, Molecular diversity, 1, 13.

In another method of the present invention, it has been surprisingly discovered that the C-7 hydroxyl group of 9-dihydro-13-acetylbaccatin III, compound 5, does not require protection prior to oxidation of the C-9 hydroxyl functionality as depicted in Scheme 2.

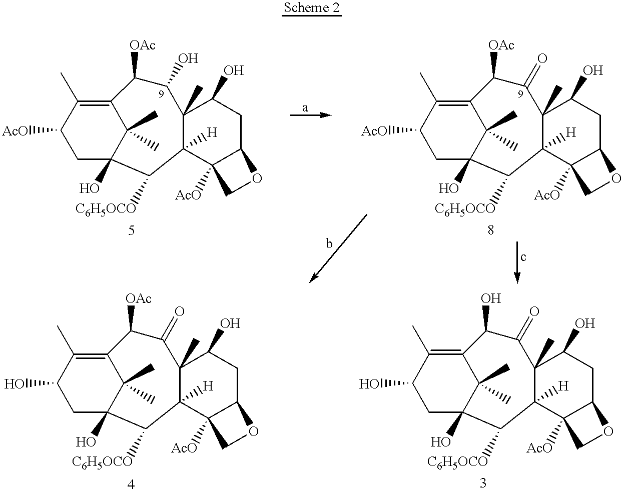

In one embodiment, the C-9 hydroxyl group of 9-dihydro-13-acetylbaccatin III, compound 5, is selectively oxidized in step a) by treatment with TPAP/NMO in acetonitrile to afford intermediate compound 8. Transformation of the C-13 acetate group into a hydroxyl group can be effected by treatment of compound 8 with methyllithium in THF or lithium hydroxide in aqueous methanol to provide baccatin III, compound 4. In another embodiment, intermediate compound 8 can be treated with hydrazine in ethanol or methanolic aqueous potassium carbonate to hydrolyze the acetate protected hydroxyl groups at C-10 and C-13 to provide 10-DAB, compound 3. In still another embodiment, 9-dihydro-13-acetylbaccatin III is treated with polymeric TEMPO (2, 2, 6, 6-tetramethyl piperidinyloxy), polysytrenedivinylbenzene methyl sulfoxide, polyethylene glycol-methylsulfoxide or (Polystyryl)trimethylammonium perruthenate (polymeric TPAP)(See for example, S. V. Ley et al, J.Chem. Soc Perkin Trans 1, 1998, 2235; S. V. Ley et al, J.Chem. Soc. Perkin Trans 1, 1997, 1907; Ley S. V. et al, J. Chem. Soc. Perkin Trans (2000), 3815; S. J. Shuttleworth et al, Synthesis 2000, 1035 (“Review, “Functionalized Polymers in Organic Synthesis; part 2), G. Bhalay et al, Synlett, 2000, 1846 (“Review, entitled, −Supported reagents: Opportunities and Limitations”) (includes work on polymeric Swern oxidations) ). As polymeric resins, the oxidative conversion from alcohol to ketone is greater than 70%. The workup is simplified by requiring only the removal of the resin by filtration. The process can be scaled into an industrial scale and the polymeric resin can be recycled several times resulting in green chemistry. The selective oxidation procedures of the present invention provide entry to compound 8, without the requirement of protecting chemistry. This eliminates a synthetic step generally required prior to oxidation of the 9-position alcohol.

The synthetic preparations provided by the invention are economical, utilize readily available starting materials, and provide high overall yields of between about 65 and 70% of the intermediates 3 and 4. The simple and elegant method of conversion from 9-dihydro-13-acetylbaccatin III, 5, to 10-DAB, 3, or baccatin III, 4, provided by the invention affords low cost highly efficient methods to produce these useful drug intermediates and analogues thereof. Thus the methods of the invention provide an entry into the efficient preparation of paclitaxel, 1, and docetaxel, 2, and analogues thereof, previously hindered by the lack of readily available starting materials.

9-Dihydro-13-acetylbaccatin III, a relatively cheap starting material provides a direct entry to baccatin III, a necessary intermediate for the semi-synthesis of paclitaxel from 10-DAB. The need to introduce an acetate group at C-10 hydroxyl group of 10-DAB, a subject of much research effort is eliminated. The preparations are high yield three step sequences, or at best two step sequence, which utilize catalytic amount or relatively inexpensive reagents. Most, if not all of the steps in the sequences can be performed under mild conditions at ambient temperature. The intermediates are easy to isolate, in most cases requiring simple extraction into a suitable organic solvent and/or filtration over an adsorbent followed by recrystallization.

Paclitaxel and docetaxel have been prepared commercially from 10-DAB and/or baccatin III by way of coupling with a suitable side chain at the C-13 hydroxyl group. Enormous effort has gone into the synthesis of the paclitaxel side chain. The more successful methods for introducing the side chain have involved esterification of a suitably protected N-benzoyl-(2R, 3S)-3-phenylisoserine such as 9 (Denis & Green J. Am. Chem. Soc., 110, (1988), 5917-5919); transesterification of oxazoline derivatives 10 (Mukayaima et al, Chem. Eur. J., 5, (1999), 121-161 and references cited therein; Kingston et al, J. Nat. Prod., 62, (1999), 1068-1071 and references cited therein); ring opening of a suitably protected β-lactam such as 11 (Holton et al, J. Am. Chem. Soc.,116, (1994), 1597-1598, Ojima et al, Tetrahedron letters, 48 (34), (1992), 6985-7012, Nicolaou et al, J. Am. Chem. Soc., 117, (1995), 624-633, Danishefsky et al, J. Am. Chem. Soc., 118, (1996), 2843-2859 and references cited therein). More recently, esterification of a chiral epoxy carboxylic acid 12 (Yamaguchi et al, Tetrahedron Lett., 39, (1998), 5575-5578 and transesterification of β-keto esters 13 have been reported (Mandai et al, Tetrahedron letters, 41, (2000), 239-242 & 243-242).

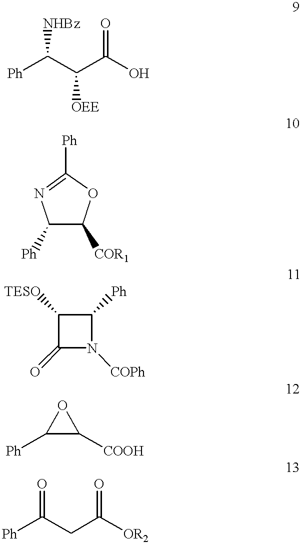

Therefore, as depicted in scheme 3, the 7-protected 9-dihydro-13 acetylbaccatin III derivatives 7 can be deacetylated selectively at C-13 with lithium hydroxide in aqueous methanol at 0° C. to provide the 7-protected baccatin III derivatives 14. The C-13 paclitaxel side chain can be introduced to compound 14 by any of the methods described above. 10

For example, 7-tesyl protected baccatin III, compound 14b when treated with dimethylsilyl sodium amide (3 eq) and the Ojima's β-lactam 11 (3.5 eq) in THF at 0° C. provides the 2′, 7-ditesyl paclitaxel, compound 15b, which when desilylated with hydrofluoric acid-pyridine affords paclitaxel 1 (Nicolaou et al, J. Am. Chem. Soc., 117, (1995), 653-659.

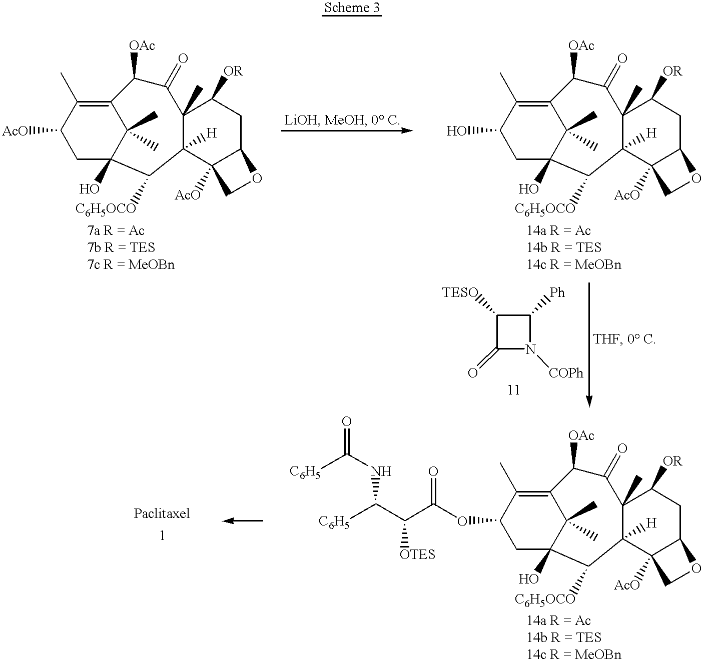

### EXAMPLES

### Example 1

9-Dihydro, 7, 13-diacetylbaccatin III 6a: To a solution of 5 and 4-dimethylamino pyridine (DMAP, 1.5 molequiv.) in dichloromethane is added acetic anhydride (1.5 molequiv). The mixture is stirred at ambient temperature for at least 2 h. The reaction is quenched with aqueous ammonium chloride (NH₄Cl) and the resulting mixture is extracted into a suitable organic solvent such as ether. The organic layer is dried with anhydrous magnesium sulfate (MgSO₄), filtered, and concentrated in vacuo. The residue is purified by flash column chromatography (Silica gel) to afford 6a in greater than 90% yield.

Suitable acyl protecting groups include: ClCH₂CO; PhCH₂O₂C (cbz); C₃H₅OCO; Cl₃CCH₂O₂C (Troc) (Holton et al, Tetrahedron Letters, 1998, 39, 2883-2886).

### Example 2

9-Dihydro, 13-acetyl, 7-O-triethylsilylbaccatin III 6b: To 5 dissolved in dry dimethyl formamide (DMF) is added imidazole (at least 3 equiv). Triethylsilylchloride (TESCl, 2.5 equiv.) is then added dropwise at room temperature. The solution is stirred at room temperature for at least 2 h. The DMF is evaporated under reduced pressure and ethyl acetate-water is added. After standard workup, the residue is purified by flash chromatography (Silica gel) affording the 7-triethylsilyl ether 6b (>80%) (Nicolaou et al, J. Am. Chem. Soc, 1995, 117, 653)

Suitable silyl ether protecting groups include: TIPS; TBDMS; (CH₃)₂i-PhSi (DMIPS); (CH₃)₂PhSi; (PhCH₂)₃Si (Holton et al, Tetrahedron Letters, 1998, 39, 2883-2886).

### Example 3

9-Dihydro, 13-acetyl, 7-O-methoxybenzylbaccatin III 6c: A solution of 5 (1 mmol) and p-methoxybenzyl alcohol (2 mmol) in dichloromethane (5 mL) is treated with Ytterbium (III) trifluroromethanesulfonate (Yb(OTf)₃) (0.05 mmol) and stirred at room temperature. Upon reaction completion as indicated by thin-layer chromatography (tic), the reaction mixture is diluted with water and the two layers are separated. The aqueous layer is extracted three times with a suitable organic solvent such as chloroform and the combined organic layers are washes with water, dried (MgSO₄), and evaporated in vacuo. The residue is purified by flash column chromatography (silica gel) affording 6c (Sharma et al, J. Org. Chem. 1999, 64, 8943-44).

Other suitable ether protecting groups include: 2-(trimethylsilyl)ethoxymethyl (SEM); THP; MOM; MEM; Benzyl; substituted benzyl such as: 2-MPM; 3,4-DMPM; 2,3,-TMPM; 3,4,5-TMPM; 2,3-DMP; 3-MPM; 2,6-DMPM (T. W. Green and P. G. M. Wuts, “Protective Groups in Organic Synthesis”, John Wiley & Sons (1999)

### Example 4

13-acetyl, 7-O— protected triethylsilylbaccatin III 7a, 7b, or 7c Solid Tetrapropylammonium perruthenate (TPAP, 5 mol %) is added in one portion to a stirred solution of the alcohol, 6a, 6b, or 6c (1 eq), 4-methylmorpholine N-oxide (NMO, 1.5 eq) and powdered 4A molecular sieves (500 mg/mmol) in dichloromethane (2 mL/mmol) or acetonitrile or a mixture of at room temperature under argon. Upon completion of reaction (tic), the acetonitrile is evaporated and the residue is dissolved in organic solvent preferably dichloromethane or ethyl acetate. The resulting solution is filtered over a pad of silica, and eluted with a suitable organic solvent. The yield of 7 is 80 to 95% (Griffith et al, Aldrichimica acta, 23, 13, 1990; Dess-Martin, J. Am. Chem. Soc., 1991, 113, 7277).

Other suitable methods for 9-OH oxidation include: Pyridinium chlorochromate (PCC) in dichloromethane, Magtrieve; Swern oxidation: Oxalyl chloride (COCI)₂, triethylamine, dimethyl sulfoxide (Mancuso A. J. and Swern D., Synthesis, 1981, 165-184); trimethylsilylhalide-oxidant (trimethylchlorocromate) (Padma S. et al European Journal of Chemistry, 1999, 375).

### EXAMPLE 5

10-Deacetylbaccatin III, Compound 3

Method A

To a solution of 7a (2 mmol) in methanol at 0° C. is slowly added an aqueous solution of K₂CO₃ (10%). The reaction mixture is stirred at 0° C. to completion (tlc). The reaction is quenched with aqueous NH₄Cl and the resulting mixture is extracted three times with organic solvent. The layers are separated, the organic layer is dried (MgSO₄), concentrated under reduced pressure, and the residue purified by flash column chromatography (silica gel) affording 10-DAB, 3 in >90% yield.

Method B

Compound 7a and hydrazine monohydrate in 95% ethanol are stirred at room temperature. The reaction progress is followed by thin-layer chromatography. Upon completion, the reaction mixture is diluted with ethyl acetate poured into saturated NH₄CL. The organic layer is separated, and washed with water and brine, dried (MgSO₄), solvent evaporated in vacuo, and the residue is purified by flash chromatography (silica gel) affording 10-DAB, 3.

Method C

The C-7 silylated compound 7b can first be deacetylated at C-10 and C-13 as in method A or method B above. After standard workup, the residue is desilylated at C-7 by treatment with HF-pyridine at ambient temperature. Upon completion (tlc), the reaction mixture is diluted with ethyl acetate and washed with 10% NaOH and brine, dried (MgSO₄), the solvent evaporated under reduced pressure, and the resulting residue purified by flash column chromatography (silica gel) affording 10-DAB, 3.

Method D

The 7-O-methoxybenzylbaccatin III 7c can first be deacetylated at C-10 and C-13 as in method A or method B above and then debenzylated according to method F.

Baccatin III, Compound 4

Method E

A solution of 13-acetyl, 7-O-triethylsilylbaccatin III 7b (0.01 mmol) in THF (0.4 mL) at 25° C. is treated with HF-pyridine (0.4 mL) and stirred for at least 2 h. The reaction mixture is diluted with ethyl acetate and washed with 10% NaOH and brine, dried (MgSO₄), and the solvent evaporated under reduced pressure. Subsequently, the residue may be deacetylated at C-13 with LiOH in aqueous methanol at room temperature and then purified by flash column chromatography (silica gel) affording baccatin III, 4.

Method F

13-acetyl, 7-O-methoxybenzylbaccatin III 7c (1 eq) and dichlorodicyanoquinone (DDQ, 1.2 eq) in dichloromethane-water; 10:1 are stirred at 20° C. Upon completion of reaction, the layers are separated. The organic layer is dried, concentrated in vacuo, and the residue purified by chromatography (silica gel). Deacetylation at C-13 to provide baccatin III, 4 is achieved as in method E above.

### Example 6

Selective Oxidation of 9-dihydro-13-acetyl Baccatin III

Method A

Tetrabutylammonium Perruthenate (TPAP)

Tetrabutylammonium perruthenate (TPAP, 41.7 mg, 0.12 mmol) was added to 9-Dihydro-13-acetyl baccatin III (1.5 g, 2.37 mmol) and 4-N-methylmorpholine (NMO, 416 mg, 3.6 mmol) in (DCM) 30 ml. The reaction mixture was stirred for 1 h at 25° C. The reaction mixture was diluted with 200 ml of ethyl acetate and filtered through a pad of silica. A second washing of the pad of silica gel with DCM gave a fraction that contains the unreacted 9-Dihydro-13-acetyl Baccatin III. The ethyl acetate and the DCM fractions were concentrated to dryness. The ethyl acetate fraction contained was purified by flash column chromatography. ¹H NMR (250 MHz) (CDCl₃) δ 1.11 (s, C-16), 1.2 (s, C17), 1.6 (s, C18), 1.88 (s, 19), 2.18 (s, C10), 2.22 (s, C13), 2.31 (s, C14), 2.24 (m, C6), 2.55 (m, C14), 4.42 (m, C13), 4.94 (d, J=7.93 Hz, C5), 5.63 (d, J=7.02 Hz, C2), 6.18 (m, C7), 6.82 (s, C10), 8.04 (o-Ph), 7.60 (p-Ph), 7.46 (m-Ph); ¹³C NMR (CDCl₃) δ 203.78 (C9), 171.29 (C10), 170.17 (C13), 169.75 (C2), 142.92 (C12), 133.73 (C1), 132.75 (p-Ph), 130.03 (o-Ph), 129.19 (q-Ph), 128.66 (m-Ph), 84.38 (C5), 81.02 (C4), 76.37 (C9), 75.70 (C20), 74.96 (C7), 72.17 (C10), 69.70 (C13), 58.57 (C3), 45.79 (C8), 43.02 (C15), 35.5 (C6), 26.66 (C14), 22.52 (C7), 15.10 (C18), 9.5 (C19); HRMS (FAB,NBA),[M+NH₄]⁺646.287, C₃₃H₄₄O₁₃ requires 646.2864

13-Acetyl baccatin III was obtained in 80% yield. The DCM fraction contained 10% 9-Dihydro-13-acetyl Baccatin III which was recycled.

Method B

1-hydroxy-1,2-benzidoxol-3(1H)-one (IBX)

A mixture of 9-Dihydro-13-acetyl Baccatin III (1000 mg, 1.58 mmol) and 1-hydroxy-1,2-benzidoxol-3(1H)-one (IBX) (1700 mg, 79 mmol) in DMSO (50 ml) was stirred at room temperature for 6 h. Water (10 ml) was added to the reaction mixture followed by extraction with dichloromethane (3×150 ml). The combined organic extract was washed with brine (150 ml), dried (MgSO₄ anhydrous), and concentrated to dryness. The residue was purified by flash chromatography (silica, hexane/ethyl acetate 1:2) and gave 13-acetyl baccatin III (695 mg, 1.11 mmol, 70%). ¹H NMR (250 MHz) (CDCl₃) δ 1.11 (s, C-16), 1.2 (s, C17), 1.6 (s, C18), 1.88 (s, 19), 2.18 (s, C10), 2.22 (s, C13), 2.31 (s, C14), 2.24 (m, C6), 2.55 (m, C14), 4.42 (m, C13), 4.94 (d, J=7.93 Hz, C5), 5.63 (d, J=7.02 Hz, C2), 6.18 (m, C7), 6.82 (s, C10), 8.04 (o-Ph), 7.60 (p-Ph), 7.46 (m-Ph); ¹³C NMR (CDCl₃) δ 203.78 (C9), 171.29 (C10), 170.17 (C13), 169.75 (C2), 142.92 (C12), 133.73 (C11), 132.75 (p-Ph), 130.03 (o-Ph), 129.19 (q-Ph), 128.66 (m-Ph), 84.38 (C5), 81.02 (C4), 76.37 (C9), 75.70 (C20), 74.96 (C7), 72.17 (C10), 69.70 (C13), 58.57 (C3), 45.79 (C8), 43.02 (C15), 35.5 (C6), 26.66 (C14), 22.52 (C7), 15.10 (C18), 9.5 (C19

Method C

2,2,6,6-tetramethyl Piperidinyloxy (TEMPO)

A mixture of 9-Dihydro-13-Acetyl baccatin III (890 mg, 1.41 mmol), tetrabutylammonium bromide (4 mol % 0.04 mmol) and TEMPO (1 mol % 1.3 mmol), and Oxone (2.2 equivalent, 1.7 mg) 10 ml of toluene were stirred at room temperature for 12 hours. Dichloromethane (90 ml) was added followed by extraction with water (2×50 ml). The organic layer was dried (MgSO₄ anhydrous) and concentrated to dryness. 13-Acetyl baccatin II was isolated in 72% yield (637.5 mg, 1.02 mmol) after flash chromatography (Hexane/Ethyl acetate 1:2).¹H NMR (250 MHz) CDCl₃ δ 8.04 (dd, J=7.17, 1.37 Hz, ortho Ph), 7.59 (t, 7.17 Hz, para Ph), 7.45 (t, J=7.17 Hz, meta Ph), 6.28 (s, H-10), 6.15 (t, J=8.88 Hz, H-13), 5.62 (d, J=7.02, H-2), 4.95 (d, J=7.93 Hz, C-5), 4.41-4.45 (m, H-7, OH), 4.32 (d, J=8.40 Hz, C20-H_{α}), 4.13 (d, J=8.24 Hz, C20-H_{β}), 3.84 (d, J=7.02 Hz, H3), 2.54 (m, H-6_{α}), 2.31 (s, C-4-OCH₃), 2.24 (s, C-13-OCH₃), 2.18 (s, C-10-OCH₃), 1.88 (s, H-18), 1.85 (m, H-6), 1.65 (s, CH₃), 1.23 (s, CH₃, H-16), 1 (s, CH₃, H-17). ¹³C NMR (CDCl₃) δ 203.77 (C-9), 171.28 (C-4-acetate), 170.17 (C-13-acetate), 169.75 (C-10-acetate), 166.92 (PhC═O), 142.92 (C-12), 133.73 (C-11), 132.74 (ortho C), 130.02 (para C), 128.65 (meta C), 84.38 (C-5), 81.02 (C-4), 76.37 (C-20), 75.69 (C-7), 72.17 (C-10), 69.70 (C-13), 45.79 (C-8), 43.02 (C-15), 35.70 (C-14), 35.55 (C-6), 26.65 (C-16), 22.52 (C-17), 21.48 (C-4-OCH₃), 21.48 (C-13-OCH₃), 20.86 (C-10-OCH₃), 15.10 (C-18), 9.48 (C-19)

Method D

Pyridinium Chlorochromate (PCC)

9-Dihydro-13-acetyl Baccatin III (1000 mg, 1.58 mmol) was refluxed with PCC in DCM under argon. The progress of the reaction was followed by TLC until completion. The reaction mixture was diluted with DCM and then filtered through a pad of silica. The titled compound was purified by flash chromatography. 13-Acetyl-Baccatin III was obtained in 65% yield (596.3 mg, 0.95 mmol) ¹H NMR (250 MHz) CDCl₃ δ 8.05 (dd, J=7.18, 1.37 Hz, ortho Ph), 7.60 (t, 7.15 Hz, para Ph), 7.44 (t, J=7.18 Hz, meta Ph), 6.27 (s, H-10), 6.16 (t, J=8.88 Hz, H-13), 5.60 (d, J=7.03, H-2), 4.95 (d, J=7.95 Hz, C-5), 4.41-4.45 (m, H-7, OH), 4.32 (d, J=8.40 Hz, C20-H_{α}), 4.13 (d, J=8.24 Hz, C20-H_{β}), 3.84 (d, J=7.02 Hz, H3), 2.54 (m, H-6_{α}), 2.31 (s, C-4-OCH₃), 2.24 (s, C-13-OCH₃), 2.18 (s, C-10-OCH₃), 1.88 (s, H-18), 1.85 (m, H-6_{β}), 1.65 (s, CH₃), 1.23 (s, CH₃, H-16), 1.11 (s, CH₃, H-17). ¹³C NMR (CDCl₃) δ 203.78 (C-9), 171.29 (C-4-acetate), 170.18 (C-13-acetate), 169.45 (C-10-acetate), 166.98 (PhC═O), 142.96 (C-12), 133.73 (C-11), 132.74 (ortho C), 130.02 (para C), 128.65 (meta C), 84.38 (C-5), 81.02 (C-4), 76.37 (C-20), 75.69 (C-7), 72.17 (C-10), 69.70 (C-13), 45.80 (C-8), 43.02 (C-15), 35.70 (C-14), 35.55 (C-6), 26.65 (C-16), 22.52 (C-17), 21.48 (C-4-OCH 21.48 (C-13-OCH₃) 20.87 (C-10-OCH₃), 15.12 (C-18), 9.47 (C-19).

### Example 7

Solution Phase Polymeric Synthesis of 13-Acetyl Baccatin III

A solution of poly(ethyleneglycol) bis (6-methylsulfinyl) hexanoate (1.7 g, 0.72 mmol) in dichloromethane (15 ml) was cooled to −50° C. oxalylchloride solution in DCM (2.0M, 0.049 ml) was added dropwise. After 15 minutes stirring at −50° C., 9-dihydro-13-acetylbaccatin III (220 mg, 0.35 mmol) in 5 ml DCM was added. The mixture was stirred for 15 minutes. Triethylamine was added and the solution kept at −45° C. for 2.0 hours before warming up to room temperature.

The reaction mixture was concentrated to 10 ml followed by the addition of diethyl ether (100 ml) to precipitate the polymer. Further precipitation was induced by cooling the ethereal solution at 4° C. After filtration, the filtrate was concentrated to give the oxidized product which was further purified by passing through a pad of silica. Further purification was done on flash column using hexane/ethyl acetate 1:2 to give 13-Acetylbaccatin III (176 mg, 0.28 mmol), 80%. ¹H NMR (250 MHz) (CDCl₃) δ 1.11(s, C-16), 1.2 (s, C17), 1.6 (s, C18), 1.88 (s, 19), 2.18 (s, C10), 2.22 (s, C13), 2.31 (s, C14), 2.24 (m, C6), 2.55 (m, C14), 4.42 (m, C13), 4.94 (d, J=7.93 Hz, C5), 5.63 (d, J=7.02 Hz, C2), 6.18 (m, C7), 6.82 (s, C10), 8.04 (o-Ph), 7.60 (p-Ph), 7.46 (m-Ph); ¹³C NMR (CDCl₃) δ 203.78 (C9), 171.29 (C10), 170.17 (C13), 169.75 (C2), 142.92 (C12), 133.73 (Cl 1), 132.75 (p-Ph), 130.03 (o-Ph), 129.19 (q-Ph), 128.66 (m-Ph), 84.38 (C5), 81.02 (C4), 76.37 (C9), 75.70 (C20), 74.96 (C7), 72.17 (C10), 69.70 (C13), 58.57 (C3), 45.79 (C8), 43.02 (C15), 35.5 (C6), 26.66 (C14), 22.52 (C7), 15.10 (C18), 9.5 (Cl9). The polymeric material was regenerated and recycled.

### Example 8

Method A

Oxidation of 9-Dihydro-7, 13-diacetoxy Baccatin III with (Polystryl)trimethylammonium Perruthenate

Dry dichloromethane (10 ml) was added to a mixture of 9-Dihydro-13-acetoxyl baccatin III (200 mg, 0.31 mmol), (Polystryl)trimethylammonium perruthenate (500 mg, 0.2 mmol) and 4-methylmorpholine-4-oxide (NMO, 54.3 mg, 49 mmol) in an Aldrich solid phase reaction flask (Aldrich). The mixture was refluxed for 12 hours. The solution was removed and the beads rinsed with dry dichloromethane (2×10 ml). The combined dichloromethane was removed in vacuo. 13-acetoxyl baccatin III was obtained in 96% yield (192 mg, 0.30 mmol). The beads were re-used with another batch of alcohol and co-oxidant and yielded 95%. ¹H NMR (250 MHz) (CDCl₃) δ 1.11 (s, C-16), 1.2 (s, C17), 1.6 (s, C18), 1.88 (s, 19), 2.18 (s, C10), 2.22 (s, C13), 2.31 (s, C14), 2.24 (m, C6), 2.55 (m, C14), 4.42 (m, C13), 4.94 (d, J=7.93 Hz, C5), 5.63 (d, J=7.02 Hz, C2), 6.18 (m, C7), 6.82 (s, C10), 8.04 (o-Ph), 7.60 (p-Ph), 7.46 (m-Ph); ¹³C NMR (CDCl₃) δ 203.78 (C9), 171.29 (C10), 170.17 (C13), 169.75 (C2), 142.92 (C12), 133.73 (C11), 132.75 (p-Ph), 130.03 (o-Ph), 129.19 (q-Ph), 128.66 (m-Ph), 84.38 (C5), 81.02 (C4), 76.37 (C9), 75.70 (C20), 74.96 (C7), 72.17 (C10), 69.70 (C13), 58.57 (C3), 45.79 (C8), 43.02 (C15), 35.5 (C6), 26.66 (C14), 22.52 (C7), 15.10 (C18), 9.5 (C19)

Method B

Oxidation with Polymer Immobilized Piperidinyl Cxyl (PIPO) TEMPO

A solution of potassium bromide (1.6 ml, 0.5M) was added to a mixture of PIPO (25 mg, 0.80 umol) and 9-Dihydro-13-acetoxyl baccatin III (100 mg, 0.158 mmol) in 20 ml of dichloromethane at 0° C. An Aqueous solution of sodium hypochlorite (NaOCl, 28 ml, 0.35M) and was added to the reaction mixture. The pH of the reaction was adjusted to 8 by NaHCO₃. Excess NaOCl was destroyed by the addition of Na₂SO₃. The reaction mixture was filtered, the residue washed with water, dried and recycled to the next reaction. The filtrate was extracted with dichloromethane (2×50 ml), dried (MgSO₄ anhydrous) and concentrated to dryness. 13-Acetyl baccatin III was obtained in 90% yield (89.3 mg, 0.142 mmol) which is used in the next reaction without further purification. ¹H NMR (250 MHz) CDCl₃ δ 8.04 (dd, J=7.17, 1.37 Hz, ortho Ph), 7.59 (t, 7.17 Hz, para Ph), 7.45 (t, J=7.17 Hz, meta Ph), 6.28 (s, H-10), 6.15 (t, J=8.88 Hz, H-13), 5.62 (d, J=7.02, H-2), 4.95 (d, J=7.93 Hz, C-5), 4.41-4.45 (m, H-7, OH), 4.32 (d, J=8.40 Hz, C20-H_{α}), 4.13 (d, J=8.24 Hz, C20-H_{β}), 3.84 (d, J=7.02 Hz, H3), 2.54 (m, H-6_{α}), 2.31 (s, C-4-OCH₃), 2.24 (s, C-13-OCH₃), 2.18 (s, C-10-OCH₃), 1.88 (s, H-18), 1.85 (m, H-6_{β}), 1.65 (s, CH₃), 1.23 (s, CH₃, H-16), 1.11 (s, CH₃, H-17). ¹³C NMR (CDCl₃) δ 203.77 (C-9), 171.28 (C-4-acetate), 170.17 (C-13-acetate), 169.75 (C-10-acetate), 166.92 (PhC═O), 142.92 (C-12), 133.73 (C-11), 132.74 (ortho C), 130.02 (para C), 128.65 (meta C), 84.38 (C-5), 81.02 (C-4), 76.37 (C-20), 75.69 (C-7), 72.17 (C-10), 69.70 (C-13), 45.79 (C-8), 43.02 (C-15), 35.70 (C-14), 35.55 (C-6), 26.65 (C-16), 22.52 (C-17), 21.48 (C-4-OCH₃), 21.48 (C-13-OCH₃), 20.86 (C-10-OCH₃), 15.10 (C-18), 9.48 (C-19)

Variations

(i). use of CuCl/PIPO as catalyst and using molecular oxygen as an oxidant and DMF as the solvents. KHCO3 can be used as buffering agent instead of NaHCO3

(ii) Use of NaOCl without KBr is another variant

(iii) Use of Oxone as an oxidant is another variant

### Example 9

10-Deacetyl Baccatin III (10-DAB III)

Hydrazine monohydrate was added to a solution of 13-Acetyl baccatin III (1000 mg, 2.56 mmol) in 95% ethanol and the mixture stirred at room temperature for 8 hours. Excess ethyl acetate (200 ml) added and the mixture was extracted with water (150 ml), brine (150 ml), and Water (150 ml). The organic layer was dried (anhydrous MgSO₄) and concentrated to dryness. The final compound was purified by flash column ethyl acetate/hexane 4:1 to yield 860.5 mg, 85%. ¹H NMR (deuterated acetone) d 8.12 (m, ortho —H), 8.01 (m, para-H), 7.56 (m, meta-H), 5.65 (d, J=7.04 Hz, C-2), 5.27 (s, H-10), 4.96 (dd, J=2.09, 9.58 Hz, C-13), 4.55 (d, J=4.63 Hz, H-5), 4.23 (m, C-7), 4.13 (d, J=7.38 Hz, H-14α), 4.04 (d, J=7.04 Hz, H-14β), 4.16 (s, OH), 2.83 (s, OH), 2.49 (m, 2H, C-14), 2.33 (m, 1H, H-6α), 1.83 (m, 1H, H-6β), 2.08 (s, 3H, C-4-OCOCH₃), 2.26 (s, OH), 2.05 (s, H-18), 1.71 (s, H-19), 1.10 (s, 3H, H-16), 1.10 (s, H-17); 13C NMR (deuterated acetone) d, 10.37 (C-19), 15.78 (C-18), 20.69 (C-17), 22.79 (C-16), 27.3 (C-4), 37.79 (C-14), 40.95 (C-15), 43.76 (C-8), 48.14 (C-3), 68.02 (C-13), 72.66 (C-10), 75.88 (C-2), 76.15 (C-20), 76.93 (C-9), 78.70 (C-1), 80.53 (C-4), 85.18 (C-5), 129.46 (meta C), 130.86 (ortho C), 134.04 (para C), 1135.76 (C-11), 143 (C-12), 170.87 (C-10), 206 (C-9)

### Example 10

Method A

Baccatin III from Acetylation of 10-deacetylbaccatin III

Acetic anhydride was added to a stirred solution of 10-Deacetyl Baccatin III (800 mg, mmol) and pyridine and stirring was continued for 10 minutes. A solution of copper sulphate was added and the mixture was extracted with DCM (3×80 ml). The organic layer was washed with brine, dried MgSO₄ anhydrous and concentrated to dryness. The residue was purified by flash chromatography (DCM/EtOAc, 7:2). Baccatin III was obtained in 80% (mg, mmol). ¹H-NMR(CDCl₃) δ 8.12 (t, J=7.05 Hz, ortho-H), 7.64 (m, 1H, para-H), 7.56 (m, 2H, meta-H), 5.66 (s, H-10), 5.60 (d, J=7.27 Hz, H-2), 5.36 (br d, 1.99 Hz), 4.96 (m, H-7), 4.92 (m, H-7), 4.56 (d, J=4.84 Hz C20-Hα), 4.56 (m, C20-Hβ), 3.5 (d, J=7.0 Hz, H-3), 2.56 (ddd, J=14.0, 9.6, 2.2 Hz, 1H, H-6), 2.3 (m, 1H, H-14), 2.27 (s, 3H, C-4-COCH₃), 1.85 (ddd, J=14.4, 10.01, 2.3 Hz 1H, H-6), 2.08 (s, 3H, C-18), 1.9 (s, 3H, C-10-OCH₃), 1.8 (s, 3H, H-19), 1.08 (s, 6H, H-16, H-17).

### Example B

Baccatin III

Butyllithium (67 ul, 2.0M) was added to a solution of 13-Acetylbaccatin III (67.6 mg, 0.1076 mmol) in 3 ml of dichloromethane at −40° C. The reaction mixture was stirred at 40° C. for 1 hour. Cold water was added and the mixture extracted with dichloromethane. The combined organic extract was washed with water, dried (MgSO₄ anhydrous), and concentrated to a residue. ¹H-NMR(CDCl₃) δ 8.12 (t, J=7.05 Hz, ortho-H), 7.64 (m, 1H, para-H), 7.56 (m, 2H, meta-H), 5.66 (s, H-10), 5.60 (d, J=7.27 Hz, H-2), 5.36 (br d, 1.99 Hz), 4.96 (m, H-7), 4.92 (m, H-7), 4.56 (d, J=4.84 Hz C20-Hα), 4.56 (m, C20-Hβ), 3.5 (d, J=7.0 Hz, H-3), 2.56 (ddd, J=14.0,9.6,2.2 Hz, 1H, H-6), 2.3 (m, 1H, H-14), 2.27 (s, 3H, C-4-COCH₃), 1.85 (ddd, J=14.4,10.01,2.3 Hz 1H, H-6), 2.08 (s, 3H, C-18), 1.9 (s, 3H, C-10-OCH₃), 1.8 (s, 3H, H-19), 1.08 (s, 6H, H-16, H-17).

### Example 11

Method A

Acetylation Reactions

A solution of 9-Dihydro-13-Acetyl baccatin III (10 g, 15.8 mmol) and Dimethylaminopyridine (DMAP) (1.22 g, 15.8 mmol) in CH₂Cl₂ (100 ml) was treated with acetic anhydride (2.5 ml). The reaction mixture was stirred at room temperature for 20 minutes followed by the addition of saturated ammonium chloride solution (500 ml). Extraction with 3×100 ml DCM followed. The combined organic layer was dried and concentrated to dryness. Yield 19 g (95%). ¹H NMR (deuterated acetone) δ 8.11 (d, J=7.05, 1.32 Hz, ortho Ph), 7.76 (t, J=7.60 Hz, para Ph), 7.55 (t, J=7.71, meta Ph), 6.16 (t, J=6.82H-13), 6.10 (d, J=11.10 Hz, H-10), 5.81 (d, J=5.95 Hz, H-2), 5.53 (d, J=7.71 Hz, H-5), 4.97 (d, J=7.81 Hz, H-9), 4.43 (dd, J=8.15, 6.37 Hz, H-7), 4.21 (d, J=7.93 Hz, C20-H_{α}), 4.14 (d, J=7.93 Hz, C20-H_{β}), 3.17 (d, J=5.73 Hz, H-3), 2.50 (m, H-14αβ), 2.48 (m, H-6α), 2.32 (s, C-4-OCH₃), 2.20 (s, C-13-OCH₃), 2.02 (s, C-10-OCH₃), 1.99 (s, H-16), 1.87 (s, H-17), 1.66 (s, H-18), 1.25 (s, H-19); 13C NMR (deuterated acetone) δ 171.25 (C-4-acetate), 170.96 (C-13-acetate), 170.34 (C-10-acetate), 170.14 (C-7-acetate), 166.57 (PhC═O), 141.47 (C-12), 136.45 (C-11), 135 (ortho Ph), 131.00 (para Ph), 129.44 (meta Ph), 84.58 (C-5), 82.46 (C-4), 78.72 (C-1), 74.61 (C-20), 74.25 (C-9), 70.49 (C-7), 48.41 (C-3), 46.14 (C-8), 43.98 (C-5), 36.98 (C-6), 35.35 (C-14), 28.71 (C-16), 23.63 (C-4-OCH₃), 23.08 (C-13-OCH₃), 21.59 (C-10-OCH₃), 21.52 (C-7-OCH₃), 20.94 (C-17), 15.31 (C-18), 13.29 (C-19); HRMS (FAB, NBA), [M+NH₄]⁺690.314, C₃₃H₄₄O₁₃ requires 690.3125

Method B

Oxidation of 9-Dihydro-7,13-diacetoxybaccatin III with TPAP

Tetrabutylammonium perruthenate (1000 mg, 0.7 mmol) was added to a solution of 9-Dihydro-13-Acetyl baccatin III (5000 mg, 4.5 mmol) 4-N-methylmorpholine (2.225 mg, 6.75 mmol) in DCM (200 ml) and stirred at room temperature. Stirring was continued for 30 minutes. The reaction was stopped by dilution with 2×1000 ml of DCM and passed through a pad of silica. The solvent was removed under vacuo to afford 4998.6 mg of 7,13-Diacetoxy baccatin III (100%). ¹H NMR (CDCl₃) δ 8.08 (d, J=7.2 Hz, 2H, Ph), 7.61 (m, 1H, Ph), 7.48 (m, 2H, Ph), 6.2 (s, H-10), 6.1 (t, J=8.37 Hz, H-2), 5.6 (d, J=7.04 Hz, H-13), 5.5 (dd, J=7.04, J=3.31 Hz, H-7), 4.9 (d, J=8.59 Hz, H-S), 4.3 (d, J=8.07 Hz, C-20α), 4.1 (d, J=8.37 Hz, C20β), 3.9 (d, J=6.8 Hz, C3H), 2.6 (m, C6), 2.2 (d, C14); ¹³CNMR (CDCl₃) δ, 11.11 (C-19), 15.08 (C-18), 20.99 (C₁₃OCH₃), 21.06 (C₁₀OCH₃), 21.43 (C₇OCH₃), 21.56 ((C₄OCH₃), 22.81 (C7), 26.74 (C14), 33.70 (C6), 43.49 (C15), 47.59 (C8), 56.43 (C3), 71.76 (C7), 74.82 (C13), 75.76 (C10), 76.66 (C2), 77.05 (C7), 77.36 (C20), 77.68 (C9), 79.14 (C1), 81.26 (C4), 84.33 (C5), 129.02 (q-Ph), 129.55 (m-Ph), 130.40 (o-Ph), 132.8 (p-Ph), 134.10 (C11), 141.75 (C12), 167.30 (C₂OCOPh), 169.23 (C₇OCOCH₃), 169.87 (C₄OCOCH₃), 170.56 ((C₁₃OCOCH₃), 170.73 ((C₁₀OCOCH₃), 202.39 (C₉OCOCH₃) HRMS (FAB, NBA), [M+NH₄]⁺688.296, C₃₅H₄₂O₁₃ requires 688.2969

Method C

Oxidation of 9-Dihydro-7,13-diacetoxybaccatin III with 1-hydroxy-1,2-benzidoxol-3(1H)-one (IBX)

A mixture of 1-hydroxy-1,2-benzidoxol-3(1H)-one (IBX) (2.227 g, 7.95 mmol), 9-dihydro-7,13-diacetoxybaccatin III (1000 mg, 1.59 mmol) in 50 ml of DMSO was stirred at room temperature for 20 h. Dichloromethane (300 ml) was added and the solution washed with water (3×90 m)l. The organic layer was dried with Magnesium sulphate anhydrous and concentrated to dryness under vacuo. The yield was 850.8 mg(85%). ¹HNMR (CDCl₃) δ 8.08 (d, J=7.2 Hz, 2H, Ph), 7.61 (m, 1H, Ph), 7.48 (m, 2H, Ph), 6.2 (s, H-10), 6.1 (t, J=8.37 Hz, H-2), 5.6 (d, J=7.04 Hz, H-13), 5.5 (dd, J=7.04, J=3.31 Hz, H-7), 4.9 (d, J=8.59 Hz, H-5), 4.3 (d, J=8.07 Hz, C-20α), 4.1 (d, J=8.37 Hz, C20β), 3.9 (d, J=6.8 Hz, C3H), 2.6 (m, C6), 2.2 (d, C14); ¹³CNMR (CDCl₃) δ, 11.11 (C-19), 15.08 (C-18), 20.99 (C₁₃OCH₃), 21.06 (C₁₀OCH₃), 21.43 (C₇OCH₃), 21.56 ((C₄OCH₃), 22.81 (C7), 26.74 (C14), 33.70 (C6), 43.49 (C15), 47.59 (C8), 56.43 (C3), 71.76 (C7), 74.82 (C13), 75.76 (C10), 76.66 (C2), 77.05 (C7), 77.36 (C20), 77.68 (C9), 79.14 (C1), 81.26 (C4), 84.33 (C5), 129.02 (q-Ph), 129.55 (m-Ph), 130.40 (o-Ph), 132.8 (p-Ph), 134.10 (C11), 141.75 (C12), 167.30 (C₂OCOPh), 169.23 (C₇OCOCH₃), 169.87 (C₄OCOCH₃), 170.56 ((C₁₃OCOCH₃), 170.73 ((C₁₀OCOCH₃), 202.39 (C₉OCOCH₃)

Method D

Oxidation of 9-Dihydro-13-acetoxylbaccatin III with 2,2,6,6-Tetramethylpiperidinyl-1-oxy (TEMPO)/oxone

A mixture of 9-Dihydro-13-Acetylbaccatin III (890 mg, 1.41 mmol), tetrabutylammonium bromide (4 mol % 0.04 mmol) and TEMPO (1 mol % 1.3 mmol), and Oxone (2.2 equivalent, 1.7 mg) 10 ml of toluene was stirred at room temperature for 12 hours. Dichloromethane (90 ml) was added followed by extraction with water (2×50 ml). The organic layer was dried (MgSO₄anhydrous) and concentrated to dryness. 13-Acetyl baccatin II was isolated in 72% yield after flash chromatography (Hexane/Ethyl acetate 1:2). (Suitable references include R. Margaritaet al, J. Org.Chem.(1997), 6974 (TEMPO-iodine oxidations, a variant of TEMPO catalysed oxidation); P. L. Anelli et al, J.Org. Chem. (1986), 2559; C. Bolm et al., Organic Letters (2000), 117.)

Method E

Oxidation of 9-Dihydro-7,13-diacetoxy Baccatin III with (Polystryl)trimethylammonium Perruthenate

Dry dichloromethane (10 ml) was added to a mixture of 9-Dihydro-7,13-diacetoxy baccatin III (200 mg, 0.31 mmol), (Polystryl)trimethylammonium perruthenate (500 mg, 0.2 mmol) and 4-methylmorpholine-4-oxide (NMO, 54.3 mg, 49 mmol) in an Aldrich solid phase reaction flask (Aldrich). The mixture is refluxed for 12 hours. The solution was removed and the beads rinsed with dry dichloromethane (2×10 ml). The combined dichloromethane was removed in vacuo. 7,13-Diacetoxybaccatin III was obtained in 96% yield (192 mg, 0.30 mmol). The beads were recycled by using another batch of alcohol and co-oxidant and yielded 95%.

Method F

Oxidation of 9-Dihydro-7,13-diacetoxy Baccatin III with 6-(Methylsulfinyl)hexanoylmethyl Polystyrene

A solution of poly (ethyleneglycol) bis (6-methylsulfinyl) hexanoate (1.7 g, 0.72 mmol) in dichloromethane (15 ml) was cooled to 0° C. and oxalyl chloride solution in DCM (2.0M) 0.049 ml was added dropwise. After 15 minutes stirring at 0° C., 9-Dihydro-7,13-diacetoxyl baccatin III (220 mg, 0.35 mmol) in 5 ml DCM was added. The mixture was stirred for 15 minutes. Triethylamine was added and the solution kept at room temperature for 1 hours before warming up to room temperature (See for example, M. Harris et al., (1998), J. Org, Chem 63 2407 and Y. Liu et al., (1996), J. Org. Chem. 61, 7856).

The reaction mixture was concentrated to 10 ml followed by the addition of diethyl ether (100 ml) to precipitate the polymer. The precipitation was accelerated by cooling to −20° C. After filtration, the filtrated was concentrated to give the oxidized product was further purified by passing through a pad of silica. Further purification was done on flash column using hexane/ethyl acetate 1:2 to give The polymeric material was regenerated by washing with dilute hydrochloric acid.

Method G

Oxidation of 9-Dihydro-7, 13-diacetoxy Baccatin III with Pyridinium Chlorochromate (PCC)

9-Dihydro-7,13-diacetoxy baccatin III (390 mg, 5.8 mmol) was added to pyridinium chlorochromate (186.0 mg, 5.8 mmol) in dichloromethane (100 ml) and stirred at room temperature for 20 h. The reaction mixture was diluted with dichloromethane (500 ml) and then filtered over a pad of silica. The pad of silica was washed with ethyl Acetate. The combined organic layer was removed in vacuo. The residue was purified by column chromatography (silica, hexane/ethyl acetate 1:1) and gave 7,13-diacetoxy baccatin III (80%). ¹H NMR (CDCl₃) δ 8.08 (d, J=7.2 Hz, 2H, Ph), 7.61 (m, 1H, Ph), 7.48 (m, 2H, Ph), 6.2 (s, H-10), 6.1 (t, J=8.37 Hz, H-2), 5.6 (d, J=7.04 Hz, H-13), 5.5 (dd, J=7.04, J=3.31 Hz, H-7), 4.9 (d, J=8.59 Hz, H-5), 4.3 (d, J=8.07 Hz, C-20α), 4.1 (d, J=8.37 Hz, C20β), 3.9 (d, J=6.8 Hz, C3H), 2.6 (m, C6), 2.2 (d, C14); ¹³CNMR (CDCl₃) δ, 11.11 (C-19), 15.08 (C-18), 20.99 (C₁₃OCH₃), 21.06 (C₁₀OCH₃), 21.43 (C₇OCH₃), 21.56 ((C₄OCH₃), 22.81 (C7), 26.74 (C14), 33.70 (C6), 43.49 (C15), 47.59 (C8), 56.43 (C3), 71.76 (C7), 74.82 (C13), 75.76 (C10), 76.66 (C2), 77.05 (C7), 77.36 (C20), 77.68 (C9), 79.14 (C1), 81.26 (C4), 84.33 (C5), 129.02 (q-Ph), 129.55 (m-Ph), 130.40 (o-Ph), 132.8 (p-Ph), 134.10 (C11), 141.75 (C12), 167.30 (C₂OCOPh), 169.23 (C₇OCOCH₃), 169.87 (C₄OCOCH₃), 170.56 ((C₃OCOCH₃), 170.73 ((C₁₀OCOCH₃), 202.39 (C₉OCOCH₃)

Method H

Deacetylation with Hydrazine Monohydrate

A solution of 7,13-Acetyl baccatin III (940 mg, 1.40 mmol) in 40 ml 95% ethanol was treated with 10 ml of hydrazine monohydrate. The reaction mixture was stirred at room temperature for 3-8 h. The reaction mixture was diluted with 100 ml of DCM and poured into a saturated solution of ammonium chloride (40 ml). The aqueous layer was extracted with 2×500 ml DCM. The combined DCM was washed with water and dried with MgSO₄ anhydrous. The DCM was removed under vacuo and the residue purified by flash column chromatography. Yield 463.25 mg, 85%. ¹H-NMR(CDCl₃) δ 8.12 (t, J=7.05 Hz, ortho-H), 7.64 (m, 11H, para-H), 7.56 (m, 2H, meta-H), 5.66 (s, H-10), 5.60 (d, J=7.27 Hz, H-2), 5.36 (br d, 1.99 Hz), 4.96 (m, H-7), 4.92 (m, H-7), 4.56 (d, J=4.84 Hz C20-Hα), 4.56 (m, C20-Hβ), 3.5 (d, J=7.0 Hz, H-3), 2.56 (ddd, J=14.0,9.6,2.2 Hz, 1H, 1H-6), 2.3 (m, 1H, H-14), 2.27 (s, 3H, C-4-COCH₃), 1.85 (ddd, J=14.4,10.01,2.3 Hz 1H, H-6), 2.08 (s, 3H, C-18), 1.9 (s, 3H, C-10-OCH₃), 1.8 (s, 3H, H-19), 1.08 (s, 6H, H-16, H-17). FAB HRMS (FAB, NBA) [M+NH4] 563.45, C₃₃H₄₂O₁₂ requires (563.454)

### Example 12

Method A

Tesylation Reactions

(a) Chlorination of Dimethylsilyl Polystyrene with 1,3,5,5-Dimethylhydantoin

A mixture of dimethylsilyl polystyrene (200 mg, 0.16 mmol), 1,3,5,5-Dimethylhydantoin (86.4 mg, 0.450 mmol) in dichloromethane were stirred for 1.5 hours. The organic liquid was removed from the resin followed by sequential wash with dichloromethane (3×6 ml) and Tetrahydrofuran (2×6 ml). The resin was dried under vacuum and used in the next reaction.

(b) Protection of Alcohol (1) with Clorodimethylsilyl Polystyrene

The Chlorodimethylsilyl polystyrene obtained in (a) above (200 mg, 0.450), imidazole (mg, 0.600 mmol) and 9-Dihydro-13-acetyl baccatin III in Dimethylformamide were stirred at room temperature for 12 hours. The organic liquid was removed.

Method B

Oxidation of the Silyloxypolymeric Protected 9-Dihydro-13-acetyl Baccatin III

Tetrabutylammoniumperruthenate (50 mg 0.14 mmol) and 9-Dihydro-13-acetyl baccatin III 1000 g) was added to the above polymer followed by 10 ml of dry DCM. The mixture was refluxed for two hours. The solvent was filtered off and the beads washed 3×50 ml. The cleaned beads were used in the cleavage of polymeric diethylsilyl polymer.

### Example 13

Protection of 9-dihydro-13-acetylbaccatin III with Methoxyethyl Silylchloride

N,N-diisopropylethylamine (0.1 ml) was added to 9-dihydro-13-acetylbaccatin f (1000 mg, 1.58 mmol) in CH₂Cl₂ 10 ml was stirred for 30 minutes. Methoxyethyl Silylchloride (0.1 ml) was added and the mixture stirred for 20 hours at ambient temperature. The reaction was diluted with CH₂Cl₂ and washed with water. The organic layer was dried (MgSO₄ anhydrous) and concentrated in vacuo. The product was purified by flash column chromatography (Hexane/ethyl acetate/Methanol 5:4:0.5). 7-Methoxyethylsilyloxy-9-dihydroacetyl baccatin III was obtained in 70% yield (715.4 mg, 1.11 mmol). ¹H NMR (400 MHz) CDCl₃, δ 8.10 (dd, J=7.01, 1.32, ortho H), 7.60 (t, J=7.49 Hz, para H), 7.51 (t, 7.49 Hz, meta H), 6.25 (d, J=12.79 Hz, H-10), 6.16 (t, J=6.13 Hz, H-13), 5.76 (d, J=5.72, H-2), 4.94 (dd, J=6.39, 6.59 Hz, C-7), 4.54 (d, J=10.90 Hz, H-9), 4.32 (d, J=8.93 Hz, H20α), 4.19 (d, J=8.85 Hz, H20β), 3.87-3.83 (ddd, J=2.98, 6.71, 7.26 Hz, OCH₂), 3.58-3.62 (ddd, J=3.1, 6.83, 10.76 Hz, CH₂O), 3.04 (d, J=5.62, Hz, H-3), 2.6 (m, H-6α), 2.27 (s, C-4-OCH₃), 2.19 (s, C-13-OCH₃), 2.17 (s, C-10-OCH₃), 2.11 (s, H-16), 1.97 (s, H-17), 1.73 (s, H-18), 1.25 (s, H-19), 0.04 (s, Si(CH₃)₃).; CDCl3, d, 172.01 (C-4-acetate), 170.11 (C-13-acetate), 169.20 (C-10-acetate), 167.22 (PhC═O), 140.79 (C-12), 134.00 (C-11), 99,29 (OCH2), 85.82 (OCH2), 84.43 (C-5), 82.23 (C-4), 78.82 (C-1), 76.67 (C-20), 73.91 (C-9), 73.29 (C-7), 67.68 (C-13), 46.41 (C-8), 43.13 (C-15), 37.33 (C-6), 35.64 (C-16), 28.39 (C-17), 23.00 (C-4-OCH₃), 21.46 (C-13-OCH₃), 18.38 (C-10-OCH₃), 14.99 (C-18), 13.07 (C-19).

### Example 14

Oxidation of 7-Methoxyethylsilyloxy-9-dihydroacetyl Baccatin III with 1-hydroxy-1,2-benzidoxol-3(1H)-one (IBX)

1-hydroxy-1,2-benzidoxol-3(1H)-one (IBX) (1000 mg, 3.96 mmol) was added to 7-Methoxyethylsilyl-9-dihydro-13-acetyl-baccatin III (1000 mg, 1.54 mmol) in 5 ml of Dimethyl Sulfoxide. The mixture was stirred at room temperature for 24 hours. Dichloromethane (100 ml) was added and the mixture extracted with water (2×50 ml). The organic layer was dried with anhydrous MgSO₄, concentrated in vacuo. The product was purified by flash chromatography (Dichloromethane/ethyl acetate 5:2) to yield 80% of 7-Methoxyethylsilyloxy-13-Acetyl baccatin III (798.76 mg. 1.23 mmol). ¹H NMR (CDCl₃) δ 8.10 (dd, J=7.05, 1.32 Hz, ortho-H), 7.64 (t, J=7.47, para H), 7.50 (t, J=7.93 Hz, meta), 6.80 (d, J=9.8 Hz, H-10), 6.08 (t, J=9.13 Hz, H-13), 5.82 (d, J=6.49, H-2), 5.15 (d, J=8.04 Hz), 4.98 (d, J=7.05 Hz, C-7), 4.45 (d, J=8.36 Hz, H20α), 4.20 (d, J=9.91, H20β), 3.72-3.76 (ddd, J=1.54, 3.97, 5.28 Hz, OCH₂), 3.49-3.50 (ddd, J=1.21, 3.74, 6.16 Hz, OCH₂), 2.98 (d, J=6.82 Hz, H-3), 2.84-2.88 (dd, J=8.48, H6αβ), 2.27 (C-4-OCH₃)2.16 (s, C-13-OCH₃), 2.15 (s, C-10-OCH3), 2.08 (s, H-16), 1.86 (s, H-17), 1.63 (s, H-18) 1.25 (s, H-19), −0.01 (Si(CH₃)₃; 13C NMR d 206.48 (C-9), 170.48 (C-4-acetate), 169.25 (C-13), 169.21 (C-10), 167.18 (C-2), 141.95 (C-12), 140.62 (C-11), 134.00 (ortho Ph), 130.30 (para Ph), 128.90 (meta Ph), 98.73 (OCH2), 86.11 (OCH2), 83.52 (C-5), 81.30 (C-4), 78.68 (C-1), 75.71 (C-20), 73.25 (C-9), 69.82 (C-7), 54.54 (C-3), 49.01 (C-8), 44.49 (C-6), 42.69 (C-14), 35.84 (C-15), 29.90 (C-16), 27.71 (C-17), 22.55 (C-4-OCH₃), 22.30 (C-13-OCH₃), 21.37 (C-10-OCH₃), 16.93 (C-18), 14.77 (C-19), −1.24 (CH₃)₃Si

### Example 15

Protection of 9-Dihydro-13-acetylbaccatin III Alcohol with Methoxyethylmethyl Chloride (MEMCl)

Methoxyethylmethyl chloride (0.2 ml, 1.68 mmol) was added to a stirred mixture 9-Dihydro-13-acetylbaccatin III (1000 mg, 1.58 mmol) and N,N-diisopropylethylamine (4 ml, mmol) in dichloromethane (80 ml). Stirring was continued at ambient temperature for 20 h. Dichloromethane (200 ml) and the mixture were extracted with water (100 ml), the organic layer was with 0.1 M HCl (200 ml) and water (100 ml). The organic layer was dried with MgSO₄ anhydrous and concentrated in vacuo to yield 7-methoxyethylmethoxy-9-dihydro-13-acetylbaccatin III 74% (535.03 mg, 0.74 mmol) following a flash chromatography (DCM/MeOH, 9:1).

### Example 16

Oxidation of 7-Methoxyethylmethyl-9-dihydro-13-Acetyl Baccatin III with 1-hydroxy-1,2-benzidoxol-3(1H)-one (IBX)

1-hydroxy-1,2-benzidoxol-3(1H)-one (IBX) (1000 mg, 3.97 mmol) was added to 7-Methoxyethylmethoxy-13-Acetyl-9-dihydrodeacetylbaccatin (500 mg, 0.69 mmol) in 30 ml of Dimethyl Sulfoxide. The mixture was stirred at room temperature for 20 hours. Dichloromethane (200 ml) was added and the mixture extracted with water (2×100 ml). The organic layer was dried with anhydrous MgSO₄, concentrated in vacuo. The product was purified by flash chromatography (Dichloromethane/ethyl acetate 5:2) to yield 100% of 7-Methoxyethylmethoxy-13-Acetylbaccatin III (498 mg. 0.68 mmol).

### Example 17

Protection of 9-dihydro-13-Acetyl Baccatin III with Chlorodimethylsilane

Chlorodimethylsilane (0.3 ml, 0.25 mmol) was added to a stirred mixture of 9-Dihydro-13-acetylbaccatin III (1000 mg, 1.58 mmol) and dimethylamino pyridine (100 mg, 1.50 mmol) in dichloromethane for 12 hours. Ethyl Acetate (200 ml) was added and the organic layer and washed with saturated ammonium chloride (150 ml). The organic layer was dried with MgSO₄ anhydrous and concentrated to dryness. 7-dimethylsilyloxy-13-9-dihydro-13-acetylbaccatin III was obtained after purification with flash chromatography (DCM/Ethyl Acetate, 5:2) 75% (516.0 mg, 0.75 mmol).

### Example 18

9-Dihydro-13-Acetyl Baccatin III with t-Butyldimethylchlorosilane (TBDMSCl)

TBDMSCl (347 mg, 0.23 mmol) was added to a stirred solution of 9-Dihydro-13-Acetyl baccatin III (500 mg, 0.79 mmol) and imidazole in DMF (20 ml). The mixture was heated at 70° C. with stirring 2 hours then cooled. Ammonium chloride solution was added and the solution extracted with DCM. The organic layer was dried with MgSO₄ and concentrated to residue. The desired compound was obtained after purification with flash column chromatography and gave 80% (470 mg, 0.63 mmol) H NMR 400MHz CDCl₃ δ 8.11 (dd, J=7.15, 1.3 Hz ortho-H), 7.62 (t, J=7.42 Hz, para-H), 7.48 (t, J=7.74 Hz, meta-H), 6.17 (t, J=8.25 Hz, H1-13), 6.03 (d, J=11.01 Hz, H-10), 5.77 (d, J=6.05, H-2), 5.38 (d, J=9.57 Hz, H-5), 4.93 (d, J=2.58 Hz, H-9), 4.55 (ddd, J=7.05, 10.13, 3.08 Hz, H-7), 4.33 (d, J=8.15 Hz, C20α), 4.19 (d, J=4.69 Hz, C20β), 3.09 (d, J=4.94, H-3), 2.54 (m, 6Hα), 2.29 (s, OC-4-OCH₃), 2.20 (s, OC-13-OCH₃), 2.12 (s, OC-4-OCH₃), 1.99 (m, H6β), 1.85 (s, H-18), 1.70 (s, H-19), 1.57 (s, H-17), 1.26 (s, H-16), 0.92 (s, SiC(CH₃)₃), 0.29 (s, SiCH₃), 0.20 (s, SiCH₃); ¹³C NMR, δ 170.55 (C-10-OCH3), 170.23 (C-13-OCH3), 167.25 (C-2), 138.71 (C-12), 135.84 (C-11), 133.82 (ortho Ph), 130.28 (para Ph), 128.80 (meta Ph), HRMS FAB (NOBA) m/e744.9, C₃₉H₅₆O₁₂Si 744.941.

### Example 19

Oxidation of 7-tButyldimethylsiloxy-9-dihydro-13-acetyl Baccatin III with 1-hydroxy-1,2-benzidoxol-3(1H)-one (IBX)

A solution of 7-tButyldimethylsilyloxy-13-acetylbaccatin III (400 mg, 0.54 mmol), 1-hydroxy-1,2-benzidoxol-3(1H)-one (IBX) (mg, mmol) in dimethylsulphoxide (10 ml) was stirred at 20° C. for 20 hours. The reaction was diluted with dichloromethane (90 ml). The organic layer was separated and washed with brine (2×90 ml), dried (anhydrous MgSO4) and concentrated to dryness. The residue was purified by flash chromatography (hexane/ethyl acetate 3:1) and gave 7-tButyldimethylsiloxy-9-dihydro-13-acetyl baccatin III 60% (241 mg, 0.0.32 mmol). ¹H NMR (CDCl₃) δ 8.07 (dd, J=7.03, 1.32 Hz, ortho Ph), 7.60 (t, J=7.43 Hz, para Ph), 7.48 (t, J=7.72 Hz, meta Ph), 6.38 (s, C-10), 6.16 (t, J=8.20 Hz), 5.69 (d, J=6.06 Hz, H-2) 4.97 (d, J=9.05 Hz, H-5), 4.04-4.44 (dd, J=7.05,3.09 Hz, H-7) 4.32 (d, J=8.20 Hz, H20α), 4.17 (d, J=4.70 Hz, H20β), 3.85 (d, J=Hz), 2.52 (m, H-14 a, 6Hα), 2.34 (s, C-4-OCH3), 2.21 (s, C-4-OCH₃), 2.15 (C-10-OCH₃), 1.85 (m, H-6β), 1.72 (s, H-16), 1.55 (s, H-17), 1.26 (s, H-18), 1.17 (s, H-19).

### Example 20

Protection of 9-dihydro-13-acetylbaccatin III with Triethylsilylchloride

Chlorotriethylsilane (0.4 ml) (357.23 mg, 2.37 mmol) was added to a stirred solution of 9-dihydro-13-acetylbaccatin III (1000 mg, 1.58 mmol) and pyridine (124.84 mg, 1.58 mmol) at ambient temperature. The reaction was allowed to warm up to room temperature. Stirring was continued for 12 hours. Copper sulphate solution (90 ml) was added to the reaction mixture followed by extraction with dichloromethane (3×90 ml). The combined dichloromethane extract was washed with brine (2×50 ml), dried (anhydrous MgSO₄), and concentrated to a residue. 7-triethylsilyloxy-9-dihydro-13-acetylbaccatin III was obtained in 60% yield. The other product of this reaction was 7, 10-di-triethylsilyloxy-9-dihydro-13-acetylbaccatin I). ¹H NMR (CDCl₃) δ 8.08 (dd, J=7.05,1.32, ortho H), 7.61 (t, J=6.48, para H), 7.48 (t, J=7.93 Hz meta H), 6.14 (t, J=8.10 Hz, H-13), 6.01 (d, J=10.47 Hz, C-10), 5.75 (d, J=5.95 Hz, H-2), 4.96 (d, J=7.95 Hz, H-5), 4.71 (d, J=10.57 Hz, H-5), 4.37 (t, J=8.91 Hz, H-7), 4.30 (d, J=5.14 Hz, H-20α), 4.12 (d, J=7.93 Hz, H-20β), 3.05 (d, J=5.72 Hz), 2.56-2.60 (ddd, J=9.02,6.39,7.70 Hz, C14αβ), 2.26 (s, C-4-OCH₃), 2.18 (s, C-13-OCH₃), 2.16 (s, C-10-OCH₃), 1.99 (s, H-17), 1.73 (s, H-16), 1.06 (t, J=7.93 Hz, CH₃CH₂), 0.82 (m, CH₃HC₂Si); ¹³C NMR(CDCl₃), δ 170.55 (C-4-acetate), 169.28 (C-13-acetate), 169.13 (C-10-acetate), 167.23 (PhC═O), 140.98 (C-12), 133.92 (C-11), 84.29 (C-5), 82.46 (C-4), 80.79 (C-1), 76.68 (C-20), 74.64 (C-9), 69.82 (C-13), 47.12 (C-3), 46.20 (C-8), 42.99 (C-15), 37.59 (C-6), 35.71 (C-14), 31.02 (C-16), 28.28 (C-17), 23.02 (C-4-OCH₃), 21.79 (C-13-OCH₃), 21.34 (C-10-OCH₃), 15.03 (C-18), 13.65 (C-19), 7.06 (SiCH₂CH₃), 5.77 (SiCH₂CH₃). (Representative examples of similar chemistry can be found in B. M. Trost et al J.Org. Chem. (1998), 4518.)

### Example 21

Oxidation of 7-triethylsilyloxy-9-dihydro-13-acetylbaccatin III

A solution of 7-triethylsilyloxy-9-dihydro-13-acetylbaccatin III (500 mg, 0.79 mmol), tetrabutylammonium perruthenate (250 mg, mmol), 4-methylmorpholine N-oxide (138 mg, 1.2 μmol), powdered molecular sieves (500 mg) in dichloromethane (30 ml) was stirred at ambient temperature for 20 hours. The reaction mixture was filtered over silica and the silica washed with 2×100 ml of dichloromethane. The combined organic filtrate was concentrated to dryness and gave 7-triethylsilyl-13-acetylbaccatin III 76% (445 mg, 0.6 mmol) after purification with flash column chromatography.

### Example 22

Protection of 9-dihydro-13-acetylbaccatin III with Triisopropylchloride Triflate

Triisopropylsilylmethanesulfonate (TIPStriflate) (1.0 ml, 37.2 mmol) was added to a stirred solution of 9-dihydro-13-acetylbaccatin III (1000 mg, 1.58 mmol), 2,6-lutideine (1.0 ml, 8.58 mmol) in 90 ml of dichloromethane at ambient temperature. Stirring was continued for 25 min. 190 ml of dichloromethane and 150 ml of copper sulphate solution (150 ml) were added. The organic phase was removed, washed with brine (150 ml), dried (MgSO₄), and concentrated to dryness. 7-triisopropylsilyloxy-9-dihydro-13-acetylbaccatin III (893 1.21 mg, mmol) was obtained after purification with flash chromatography (hexane:ethyl acetate 2:1) in 76% yield. (A general reference for protection with triisopropylsilyl groups can be found in C. Rucker, Chem. Rev. (1995), 1009.)

### Example 23

Oxidation of 7-triisopropylsilyloxy-9-dihydro-13-acetylbaccatin III

A solution of 7-triisopropylsilyloxy-9-dihydro-13-acetylbaccatin Ii (400 mg, 0.63 mmol), tetrabutylammonium perruthenate (200 mg, mmol), 4-methylmorpholine-N-oxide (NMO) (147 mg, 1.26 mmol), and powdered molecular sieves (500 mg) in dichloromethane 20 ml was stirred at room temperature for 20 hours. The mixture was filtered over silica and filtrate concentrated to a residue. 7-triisopropylsilyloxy-13-acetylbaccatin III 70% (3280 mg) was isolated after purification with flash column chromatography (hexane: ethyl acetate).

### Example 24

Protection of 9-dihydro-13-acetylbaccatin III with Methoxyphenylbromide

A mixture of 9-dihydro-13-acetylbaccatin III (200 mg, 0.31 mmol), methoxybenzyl alcohol (101 mg, 0.5 mmol) in dichloromethane (10 ml) was reflux for 2 h. The reaction mixture was cooled to room temperature. Dichloromethane was added and the organic layer separated. The organic layer was dried (MgSO₄), concentrated to dryness. The residue was chromatographed (flash column, hexane/ethyl acetate 3:1) and gave 7-Methoxybenzyloxy-9-dihydro-13-acetylbaccatin III 60% (136 mg, 0.68 mmol). (For exemplary reaction conditions, see, G. V. M. Sharma et al, J. Org. Chem. (1999), 8943.)

### Example 25

Protection of 9-dihydro-13-acetylbaccatin III with Benzoic Anhydride

9-dihydro-13-acetylbaccatin (500 mg, 0.59 mmol) was added to a stirred solution of benzoylchloride (125 mg, 0.89 mmol) and dimethylamino pyridine (122 mg, 1 mmol) in dichloromethane (20 ml) at 20° C. The mixture was stirred at 20° C. for 6 hours. Water was added and the organic layer was separated. The aqueous phase was extracted with dichloromethane (3×50 ml). The combined organic extract was washed with brine, dried (anhydrous MgSO4), and concentrated to a residue. The residue was purified by flash chromatography (hexane: ethyl acetate 2:1) and gave 7-benzyoloxy-9-dihydro-13-acetylbaccatin III 69% (298 mg, 0.41 mmol).

Protection of 9-dihydro-13-acetylbaccatin III with Polymeric Trityl Chloride

9-dihydro-13-acetylbaccatin III (500 mg, 0.79 mmol) is added to a pre-swollen 2-chlorotritylchloride resin (200 mg, 1.3 mmol/g loading) and diisopropylethyl amine (DIEA) (0.3 ml, 1.58 mmol) in DCM (30 ml) and the mixture reflux. The progress of the reaction is followed by TLC (hexane:ethyl acetate 1:2). The resin is filtered and followed by washing with THF×2, DCM×2, MeOH×2, and DCM×2.7-O-polymer bound-9-dihyro-13-acetylbaccatin III is oxidized by methods described herein. Suitable methods for protection by trityl chloride are generally known. See for example, Z. Zhu and B. McKittrick, Tetrahedron Letters (1998), 7479, J. J. McNally et al, Tetrahedron Letters (1998), 967 or B. M. Trost et al J.Org. Chem. (1998), 4518. See also, S. Yoo et al, Tetrahedron Letters (2000), 6415 for vinyl derivatives.

Oxidation of 7-O-polymer Bound-9-dihyro-13-acetylbaccatin III with TPAP

7-O-polymer bound-9-dihyro-13-acetylbaccatin III obtained from the above reaction is added to a stirred mixture of tetrabutylammonium perruthenate (200 mg, 0.56 mmol), 4-methylmorpholine N-oxide (132 mg, 1.13 mmol), in dichloromethane (30 ml) the mixture refluxed. The progress of the reaction is followed by TLC. On completion of the reaction, the resin is washed with THF×2, DCM×2, MeOH×2, and DCM×2. The resin is cleaved with 2 ml of 7:1:2 DCM:MeOH:TFA to generate 13-Acetylbaccatin III.

Acetylation of 9-dihydro-13-acetylbaccatin with PEG Supported Polystyrene Acid Chloride

9-dihyro-13-acetylbaccatin (500 mg, 0.79), diisopropylethyl amine (0.3 ml 01.58 mmol), dimethylamino pyridine (10 mg, 0.08 mmol) dissolved is added to a suspension of PEG supported acid chloride resin (0.5 g, 0.3 mmol/g loading). The mixture is stirred and the progress of the reaction followed by TLC. Wash the resin with DCM, DCM/MeOH (2:1), MeOH, and dried. The 9-dihydro-13-acetylbaccatin is subjected to oxidation by TPAP/NMO or IBX, or TEMPO or polymeric TEMPO (as described above, reference citations included). The resin is cleaved by hydrazinolysis to give 10-deacetylbaccatin III.

Carboxypolystyrene acid chloride is another variant of resins used in the acetylation reaction.

One of ordinary skill in the art will appreciate further features and advantages of the invention based on the above-described embodiments. Accordingly, the invention is not to be limited by what has been particularly shown and described, except as indicated by the appended claims. All publications and references cited herein, including those in the background, are expressly incorporated herein by reference in their entirety.

What is claimed is:

1. A compound having the formula 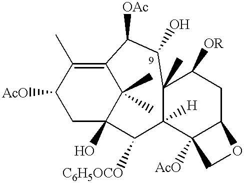
wherein R is a polymeric protecting group or an acetate group.

2. The compound of claim 1, wherein R is an acetate group.

3. The compound of claim 1, wherein the polymeric protecting group is selected from the group consisting of polystyrenebutyldimethylsilylchloride, 4-(bromomethyl)phenoxymethyl polysytrene, polyethylene glycol supported acid chloride polysytrene, carboxypolystyrene acid chloride and polymeric trityl chloride.

4. A compound having the formula 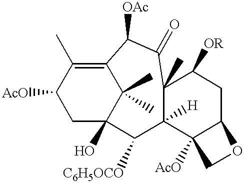
wherein R is a polymeric protecting group or an acetate group.

5. The compound of claim 4, wherein R is an acetate group.

6. The compound of claim 4, wherein R is a polymeric protecting group selected from the group consisting of polystyrenebutyldimethylsilylchloride, 4-(bromomethyl)phenoxymethyl polysytrene, polyethylene glycol supported acid chloride polysytrene, carboxypolystyrene acid chloride and polymeric trityl chloride.

7. A compound having the formula

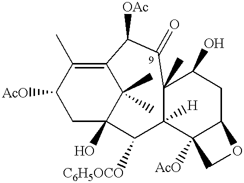

8. A compound having the formula

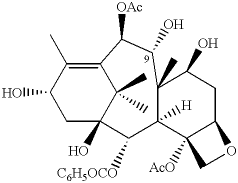

9. A method for the preparation of baccatin III, comprising the steps of:
a) selectively protecting the C-7 hydroxyl group of 9-dihydro-13-acetylbaccatin III as either an acetate group or with a polymeric protecting resin;
b) selectively oxidizing the C-9 hydroxyl group with an oxidizing agent; and
c) selectively deprotecting the protected C-7 hydroxyl group after oxidation to provide baccatin III.

10. The method of claim 9, wherein the polymeric protecting resin is selected from the group consisting of polystyrenebutyldimethylsilylchloride, 4-(bromomethyl)phenoxymethyl polysytrene, polyethylene glycol supported acid chloride polysytrene, carboxypolystyrene acid chloride and polymeric trityl chloride.

11. The method of claim 9, wherein the oxidizing agent is polymeric tetrabutylammonium perruthenate or IBX.

12. A method for the preparation of 10-deacetylbaccatin III, comprising the steps of:
d) selectively protecting the C-7 hydroxyl group of 9-dihydro-13-acetylbaccatin III as an acetate group or with a polymeric protecting resin;
e) selectively oxidizing the C-9 hydroxyl group with an oxidizing agent; and
f) selectively deprotecting the protected C-7 hydroxyl and C-10 hydroxyl groups after oxidation to provide 10-deacetylbaccatin III.

13. The method of claim 12, wherein the polymeric protecting resin is selected from the group consisting of polystyrenebutyldimethylsilylchloride, 4-(bromomethyl)phenoxymethyl polysytrene, polyethylene glycol supported acid chloride polysytrene, carboxypolystyrene acid chloride and polymeric trityl chloride.

14. The method of claim 12, wherein the oxidizing agent is polymeric tetrabutylammonium perruthenate or IBX.

15. A method for the preparation of the compound having the formula 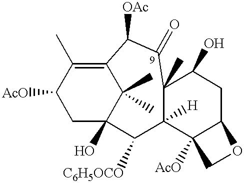
from 9-dihydro-13-acetylbaccatin III, comprising the step of:
treating 9-dihydro-13-acetylbaccatin III with an oxidizing agent such that only the C-9 hydroxyl group of 9-dihydro-13-acetylbaccatin III is oxidized to the corresponding ketone.

16. The method of claim 15, wherein the oxidizing agent is tetrabutylammonium perruthenate.

17. The method of claim 15, wherein the oxidizing agent is 2,2,6,6-tetramethylpiperidinyl-1-oxy.
